(19) United States
(12) Patent Application Publication
(10) Pub. No.: US 2015/0342968 A1
(43) Pub. Date: Dec. 3, 2015

(54) STABLE PHARMACEUTICAL COMPOSITION FOR TREATING OSTEOPOROSIS

(71) Applicant: LANDSTEINER SCIENTIFIC S.A. DE C.V., Distrito Federal (MX)

(72) Inventors: Maria Lourdes García Mondragón, Colonia Suteym (MX); Alejangro Reyes Santiago, Huixquilucan (MX)

(73) Assignee: LANDSTEINER SCIENTIFIC S.A. DE C.V., Distrito Federal (MX)

(21) Appl. No.: 14/647,981

(22) PCT Filed: Oct. 30, 2013
(86) PCT No.: PCT/MX2013/000130

§ 371 Date: May 28, 2015

(30) Foreign Application Priority Data
Dec. 3, 2012 (MX) MX/A/2012/014071

(51) Int. Cl. A61K 31/675; A61K 9/28; A61K 9/20; A61K 31/593

(52) U.S. Cl. CPC A61K 31/675; A61K 31/593; A61K 9/28; A61K 9/2095

(57) ABSTRACT

This invention provides pharmaceutical compositions which increase the bioavailability of risedronate sodium and provides vitamin D3 with greater stability; these compositions are useful in the inhibition of bone resorption.

### FIELD OF THE INVENTION

This invention refers to stable compositions which consist of therapeutically effective doses of risedronate and cholecalciferol used to inhibit bone resorption, especially osteoporosis.

### BACKGROUND

Bone resorption leads to a reduction in bone mass, causing diseases such as postmenopausal osteoporosis, osteoporosis in males, glucocorticoid-induced osteoporosis and Paget's disease (which involves the bone destruction and abnormal regeneration, causing deformity), among others.

The appearance of osteoporosis increases with age and therefore there are greater complications associated with bone fracture, as well as higher medical costs. The treatment of bone resorption and related bone diseases requires medications which use a bisphosphonate as an active ingredient, which inhibits bone resorption. The bisphosphonates used includes the salts of the alendronic, pamidronic, risedronic, ibandronic and zolendronic acids.

It has been reported that the administration of bisphosphonates is associated with hypocalcemia, since they inhibit bone resorption by osteoclasts, preventing calcium leakage from the bone, and consequently reducing calcium concentration in the blood. The reduction of calcium in the blood produces a homeostatic increase in the level of parathyroid hormone in the blood, which is dangerous for a patient receiving treatment for vitamin D deficiency, parathyroid dysfunction, poor liver function or Paget's disease, requiring the appropriate intake of calcium and vitamin D before and during the bisphosphonate treatment.

Given that a bisphosphonate treatment increases the calcium requirement, calcium absorption must be increased in order to achieve an effective osteoporosis treatment. Therefore, the level of vitamin D in the blood is an important factor.

Based on the foregoing, vitamin D intake is recommended during bisphosphonate treatment. The normal recommended dose is between 400 and 800 UI.

Vitamin D increases calcium absorption in the small intestine, and plays an important role in cellular differentiation in bone and in the generation of high quality bone matrix in the osteoblasts. It is important to maintain proper levels of vitamin D or its metabolites in the blood in order to improve bone metabolism as an effective treatment against osteoporosis.

The active form of vitamin D3 is chemically unstable in terms of temperature, oxygen and light, and therefore requires refrigeration, protection from light and the substitution of 02 by an inert gas. It is an insaponifiable heterolipid from the steroid group, and the active forms of Vitamin D3 is the 1αa-hydroxycholecalciferol, 1α, 25—dihydroxy-cholecalciferol or 1 α24-dyhydroxycholecalciferol, also known as calcitriol, which is converted in the kidneys from the 25-hydroxycholecalciferol or calcitriol in the circulating blood, which in turn is converted in the liver from vitamin D3. It functions to increase calcium and phosphorus absorption in the intestine, induce the formation of osteoclasts for bone resorption (increases the calcium concentration in the blood) and reduce the production of parathyroid hormone (PTH).

Risedronate is a pyridinyl bisphosphonate that binds to the bone hydroxyapatite and inhibits bone resorption by osteoclasts, increases bone mass and skeletal biomechanical resistance, depending on the dose. Its activity was confirmed by measuring biochemical markers of bone remodeling.

Risedronate absorption is relatively quick after administering an oral dose (Tₘₐₓ˜1 hour), and its speed is independent of the dose (single dose of 2.5 to 30 mg; multiple dose of 2.5 to 5 mg daily, and up to 50 mg weekly). It has an average oral bioavailability of 0.63%, and is reduced with food; it produces similar effects in both men and women. The mean volume of distribution at steady state equilibrium is 6.3 L/kg in humans. The plasmatic protein binding is 24%. There is no evidence of systemic metabolism of risedronate sodium. One half of the absorbed dose is excreted in the urine during the first 24 hours, and 85% of an injected dose is recovered in the urine after 28 days.

It is prescribed in the treatment of post-menopausal osteoporosis, osteoporosis in males, glucocorticoid-induced osteoporosis, and Paget's disease. Side effects include: known hypersensitivity to risedronate sodium or to any of its excipients, hypocalcemia and serious renal failure.

With regard to technique, various combinations have been described, including risedronate and vitamin D; the application MX/a2012/002786, PHARMACEUTICAL COMPOSITIONS INCLUDING HIGH DOSE BISPHOSPHONATE DERIVATIVES AND CHOLECALCIFEROL, presents a pharmaceutical composition for the prevention or treatment of osteoporosis that may be administered once a month; the pharmaceutical composition includes risedronic acid (or its salt) and cholecalciferol, in a high dose. The composition includes: (a) granules containing cholecalciferol adsorbed in microcrystalline cellulose, the quantity of cholecalciferol is 24,000 to 50,000 UI; and one or more excipients chosen from tocopheryl acetate, butylhydroxytoluene, and butylhydroxyanisole as a first stabilizing agent; and a binding agent in ethanol or an aqueous ethanol solution, mannitol as a second stabilizing agent, and risedronic acid or its salt.

The Mexican application PA/a/2004/004807, COMPOSITIONS AND METHODS TO INHIBIT BONE RESORPTION, describes the compositions and methods used to prevent, inhibit, reduce or treat conditions and diseases associated with abnormal bone resorption in mammals, such as osteoporosis; the modalities of the compositions of the invention include a pharmaceutically effective quantity of alendronate and vitamin D3 prepared for a once-a-week dose; the compositions and methods of the invention provide vitamin D nutrition during the treatment with bisphosphonate to facilitate normal bone formation and mineralization, while reducing to a minimum the complications associated with vitamin D deficiency or the potential thereof, such as hypocalcemia and osteomalacia; it also describes methods for manufacturing compositions of the invention herein, for measuring the stability and degradation of the compositions, and for measuring vitamin D levels in blood plasma.

The medication known as Actonel Plus Ca & D includes enteric coated capsules containing 35 mg of risedronate, and effervescent granules which contain 1 g of calcium carbonate and 880 UI of vitamin D3. The capsules include lactose monohydrate, microcrystalline cellulose, crospovidone A, magnesium stearate; the enteric coating includes: hypromellose, macrogol, hydroxypropylcellulose, silicon dioxide, titanium dioxide (E171), red and yellow iron oxide (E172); the effervescent granules include: anhydrous citric acid, malic acid, gluconolactone, maltodextrin, sodium cyclamate, sodium saccharin, sorbitol E420, Mannitol E421; it also includes other excipients such as dextrin, acacia, natural lemon oil, lemon scent, rice starch, potassium carbonate, tocopheryl, hydrogenated soybean oil, gelatin, sucrose, corn oil.

Actonel is prescribed in the treatment of postmenopausal osteoporosis to reduce the risk of vertebral fractures. It is only prescribed for patients whose assessment indicates that the quantity of calcium and vitamin D3 that is included will provide their adequate supplement.

The weekly unit of Actonel Plus Ca & D consists of 1 coated tablet with 35 mg resonate and 6 packets of calcium and vitamin D3. The adult recommended dose is one 35 mg tablet of risedronate on the first day, followed by 1 packet of calcium, vitamin D3 and one more each day for 5 days. This 7-day sequence is repeated each week. The absorption of risedronate sodium is affected by foods; , in order to ensure the proper absorption, patients must take the tablet before breakfast: at least 30 minutes prior to the first food, medicine or drink (except water) of the day.

The tablet must be swallowed whole and may not be sucked or chewed. To facilitate the tablet's entry into the stomach, the patients must not lie down for 30 minutes after taking the tablet and must remain standing. The packet of calcium and vitamin D3 must be taken daily 6 days a week, starting the day after the risedronate tablet is taken. The contents of the packet are emptied into a glass of water, and must be stirred and drunk immediately.

The U.S. Pat. No. 7,473,684 dated Jan. 6, 2009, and U.S. Pat. No. 8,093,228 dated Jan. 10, 2012, both named: Formulation of Bisphosphonate, describe a bisphosphonate which includes risedronate for the treatment of osteoporosis, which is formulated with a quantity of anti-foaming agent that is effective in reducing the formation of foam in the stomach, reducing reflux and reduced esophageal irritation, achieving greater patient compliance.

The US patent application serial no. 2010/0048511 A1. COMPLEX FORMULATION TO PREVENT OR TREAT OSTEOPOROSIS INCLUDING THE SOLID DISPERSION OF VITAMIN D OR ITS DERIVATIVES AND BISPHOSPHONATE, describes a solid dispersion with vitamin D or a derivative thereof and a cyclodextrin, as well as a method for preparing the complex formulation. The complex formulation can maintain a constant therapeutic level of vitamin D or a derivative thereof via its improved stability, while improving patient compliance by minimizing discomfort and side effects when administered.

The publication WO2010090614 A1, PHARMACEUTICAL FORMULATION COMPRISING RISEDRONATE, CALCIUM CARBONATE AND VITAMIN D3 IN ONE COMBINED DOSE; the invention relates to pharmaceutical compositions which include the combination of the active ingredients in a single dose in order to increase patient compliance for the treatment of diseases and conditions associated with abnormal bone resorption. The combination includes a calcium salt, vitamin D and risedronate or pharmaceutically acceptable salt, derivative or hydrate thereof.

Therefore, it is plausible to have a composition that would coordinate the benefits of risedronate and vitamin D3, without involving the drawbacks of the compositions that are available so far.

### OBJECT OF THE INVENTION

The object of the present invention is to provide pharmaceutical compositions which increase the bioavailability of risedronate sodium, since it has very low absorption (0.63%). We propose increasing the bioavailability of risedronate to obtain a lower dose with the same effect.

A second object of the invention is to provide a shield for the Vitamin D3, by treating the Vitamin D3 powder to increase its stability through the use of suitable excipients and antioxidants containing an enteric coating.

Therefore, it provides a composition which can maintain Vitamin D3 in a stable form through a shielding process using excipients to form a co-processed excipient and to provide a transmembrane carrier, increasing the bioavailability (greater than 0.63%) of risedronate sodium and vitamin D.

### DESCRIPTION OF THE INVENTION

The pharmaceutical compositions of the present invention include a granulated core containing risedronate or a pharmaceutically acceptable salt thereof and at least one excipient, which acts as an antiresorptive agent on bone that is useful in treating osteoporosis; cholecalciferol, vitamin D3 with sprayed shielding on the risedronate granules, a first coating of the risedronate granules sprayed with the shielded cholecalciferol; pharmaceutically acceptable excipients for the preparation of a tablet made from risedronate granules sprayed with shielded cholecalciferol and coated; and an enteric coating on the tablet. The composition comprises between 10 to 40% of the weight of the risedronic acid composition or its pharmaceutically acceptable derivatives, its salts, anfiolates and hydrates; between 0.001 to 0.05% of the weight of the composition of cholecalciferol, Vitamin D3; from 50 to 80% of the weight of the composition of at least one emulsifier, from 2 to 6% of the weight of the composition of at least one disintegrating agent, from 2 to 10% of the weight of the composition of at least one diluting agent, 0.1 to 2% by weight of the composition of at least one binding agent, from 0.2 to 0.5% of the weight of the composition of a first enteric coating, from 3 to 5% of the weight of the composition of a second enteric coating, from 0.25 to 5% of the weight of the composition of at least one lubricating agent and a sufficient amount of one or more solvents or solvent mixture as the dissolution medium.

Wherein the diluent is selected from the group including cellulose PH 102, microcrystalline cellulose PH 101, Ludipress, Prosolv 90; the disintegrating agent is selected from the group consisting of croscarmellose sodium, crospovidone, and sodium starch glycolate; the binder is selected from the group consisting of polyvinylpyrrolidone K 30, povidone PK 90, copovidone, hydroxypropyl methylcellulose, ethyl cellulose; the emulsifying agent is selected from the group consisting of Phosal 50 PG phosphatidylcholine in soy lecithin, propylene glycol, sunflower mono- and diglycerides and ascorbyl palmitate; the composition comprising an enteric coating is selected from NS Enteric and sodium alginate, Acryl-eze™, methacrylic acid copolymer, Sureteric®, polymethacrylates, triethylcitrate, triacetin, and polyethylene glycol; and solvents are selected from purified water, ethanol, propanol and mixtures thereof.

Preferably, the composition of the risedronate granule includes risedronate sodium, a disintegrating agent selected from the group consisting of croscarmellose sodium, microcrystalline cellulose and povidone K30.

The cholecalciferol shield consists of an emulsifier selected from the group comprised of Phosal, phosphatidylcholine in soy lecithin, propylene glycol, sunflower mono- and diglycerides and ascorbyl palmitate and ethanol as solvent. This solution is sprayed on the granulated risedronate sodium, and subsequently coated with a suspension of NS Enteric coating.

The tablet also includes one or more of the following excipients selected from croscarmellose sodium, microcrystalline cellulose, povidone K30, Phosal and NS Enteric agent and a lubricant, such as magnesium stearate.

The coating consists of a methacrylic acid copolymer (in Acryl-eze™), which gives the product gastro-resistant properties.

The following tables show examples of the compositions of this invention.

TABLE 1

| Component | Quantity | |
|---|---|---|
| Risedronate sodium | 35.00 | mg |
| Vitamin D3 | 2000 | UI |
| Microcrystalline cellulose PH | 132.95 | mg |
| 102 | | |
| Croscarmellose sodium | 9.00 | mg |
| Polyvinylpyrrolidone K 30 | 10.00 | mg |
| Phosal 50 PG | 1.00 | mg |
| NS Enteric | 10.00 | mg |
| Magnesium stearate | 2.00 | mg |
| Ethanol | 48.62 | mg |
| White Acryl-eze ™ | 14.00 | mg |
| Triethyl citrate | 1.40 | mg |
| Opadry ® FX | 0.60 | mg |
| Purified water | 84.00 | mg |
| TOTAL | 216.00 | mg |

TABLE 2

| Component | Quantity | |
|---|---|---|
| Risedronate sodium | 30.00 | |
| Vitamin D3 | 2500 | UI |
| Ludipress | 150 | mg |
| Crospovidone | 12.0 | mg |
| Hidroxymethylcellulose | 15.00 | mg |
| Propylene glycol | 1.5 | mg |
| NS Enteric | 10.00 | mg |
| Magnesium stearate | 2.00 | mg |
| Ethanol | 48.62 | mg |
| Purified water | 84.00 | mg |
| TOTAL | 220.5 | mg |

TABLE 3

| Component | Quantity | |
|---|---|---|
| Risedronate sodium | 25.00 | mg |
| Vitamin D3 | 2000 | UI |
| Prosolv 90 | 150 | mg |
| Croscarmellose sodium | 9.0 | mg |
| Polyvinylpyrrolidone K 30 | 10.00 | mg |
| Phosal 50 PG | 1.00 | mg |
| Sodium alginate | 10.00 | mg |
| Magnesium stearate | 2.00 | mg |
| Ethanol | 48.62 | mg |
| Methacrylate copolymer | 21.00 | mg |
| Triacetin | 2.1 | mg |
| Purified water | 84.00 | mg |
| TOTAL | 207.05 | mg |

The compositions presented above are prepared using the procedure described below for the composition in Table 1:

### Preparation of The Granule Composition:

Weigh, mix and granulate risedronate sodium, croscarmellose sodium, microcrystalline cellulose PH 102 and polyvinylpyrrolidone K 30 with purified water.

Sift using No. 14 mesh and dry in oven until less than 3.0% moisture is reached.

### Preparation Of Vitamin D3 Shield

Dissolve vitamin D3 in Phosal 50 PG and subsequently dilute with ethyl alcohol, mix until it forms a clear, yellow solution.

Introduce the dry risedronate granules into a fluid bed basin, and permeate with the vitamin D3 spray solution at a temperature between 25° to 30° C.

### Granule Enteric Coating

Prepare a suspension of NS Enteric in purified water at 10% solids, stir until completely incorporated and sift using No. 50 mesh.

Spray the above suspension in the granules at a temperature of 25° to 35° C., dry the granules in a fluid bed and sift using No. 18 mesh.

### Preparation of the Core

Sift using No. 20 mesh: microcrystalline cellulose PH 102, croscarmellose sodium and mix with the granules from step No. 6 for 5 minutes. Lubricate with magnesium stearate, previously sieved through No. 30 mesh, for 3 minutes.

Compress in 4 mm hexagonal press at 200 mg ±5% of weight.

### Enteric Coating of the Tablet-Core and Shielded Vitamin D3

Prepare a coating suspension with Acryl-eze™ and triethyl citrate in purified water at 20% solids.

Coat the tablets with the Acryl-eze™ suspension at a temperature of 30° to 35° C. in a perforated drum.

Prepare the coating suspension with Opadry® FX in purified water to 6.5% solids.

Coat the tablets with the Opadry® FX suspension at a temperature of 38° to 43° C. in a perforated drum.

Two stability tests of vitamin D3 and risedronate sodium were performed at different conditions by subjecting the tablets that were prepared according to the composition in Table 1. The conditions were as follows:

### 40° C./75% RH

### 30° C./65% RH

### Cycled (40° C./75% RH-cooling).

The results of the tests that were performed are presented below:

### Tests:

### Dissolution Acid Stage

| | | RISEDRONATE |
|---|---|---|
| | | SODIUM |
| | | Acid Stage |
| Specification | Sample | % Dissolved |
| No more than 10.0% | S1 | 0.56% |
| | S2 | 0.80% |
| | S3 | 0.95% |
| | S4 | 1.19% |
| | S5 | 0.71% |
| | S6 | 0.97% |
| | Mean | 0.86% |
| | Maximum | 1.19% |
| | Minimum | 0.56% |
| | % RSD | 0.00% |

The product complies with the gastro-resistant stage, because it has an enteric coating to protect the mucous membranes while it passes through the gastrointestinal system.

### Dissolution Buffer Stage

pH = 6.8

| TIME (MIN) | Risedronate Sodium 35 mg |
|---|---|
| 5 | 94.08 |
| 10 | 100.93 |
| 15 | 100.84 |
| 20 | 100.94 |
| 30 | 100.12 |
| 45 | 100.48 |

It is evident that the risedronate sodium dissolves quickly in a medium with pH 6.8, obtaining more than 100% in the first 10 minutes.

### Evaluation Of The Stability Of Vitamin D3

Two batches of the composition were subjected to different conditions to evaluate the stability of vitamin D3 and risedronate sodium. The conditions were the following: 30° C./65% RH, 40° C./75% RH and Cycled 40° C./75% RH-Cooling (2°-8° C.). The samples were submitted for 4 weeks and the following results were obtained.

### Results of Test 1

Conditions

| Week | 30° C./65% RH | 40° C./75% RH | Cycled |
|---|---|---|---|
| 0 | 100.00% | 100.00% | 100.00% |
| 1 | 102.02% | 98.34% | 98.69% |
| 2 | 99.20% | 98.12% | 97.90% |
| 4 | 96.39% | 96.03% | 96.09% |

### Results of Test 2

Conditions

| Week | 30° C./65% RH | 40° C./75% RH | Cycled |
|---|---|---|---|
| 0 | 100.00% | 100.00% | 100.00% |
| 1 | 97.34% | 99.62% | 95.55% |
| 2 | 96.93% | 97.80% | 96.31% |
| 4 | 94.79% | 96.32% | 94.79% |

### Evaluation of the Stability of Risedronate Sodium

| | Test 1 | Test 2 |
|---|---|---|
| Initial | 102.55% | 100.99% |
| Final | 100.66% | 96.75% |

It is evident that vitamin D3 shows a slight decrease (5.96%) in the rating over the four weeks in which it was subjected to different conditions, and therefore we conclude that vitamin D3 achieved stabilization with the proposed manufacturing process.

On the other hand, it has been proven that the formulation meets the dissolution specifications in the acid stage and in the buffer stage.

The objective of the combination of sodium risedronate and vitamin D3 is to seek a synergetic relationship for the treatment of osteoporosis, the risedronate sodium inhibits bone resorption and osteoblast activity and bone mineralization by Vitamin D3 aids in calcium absorption; this is achieved in a single pharmaceutical form containing both active ingredients in a formulation which stabilizes vitamin D3, since it is chemically unstable in the environment. The risedronate sodium is a soluble but waterproof product; it has an added solubilizing agent that acts as a transmembrane carrier to increase the low bioavailability of the risedronate sodium, which will produce pharmacological effects in less time than the products currently available.

The tests performed on the product show that it was possible to stabilize the vitamin D3 without modifying the stability of the risedronate sodium, it meets pharmacopeial dissolution specifications for risedronate sodium, and a gastro-resistance test was performed to ensure that it does not come into contact with the mucous membranes, since that would cause severe irritation. In the buffer stage it was shown that the product is bioavailable in less than 30 minutes, to ensure that no problems will occur in the dissolution of the risedronate in the intestine, where the absorption will take place.

In conclusion, the pharmaceutical composition of risedronate sodium/vitamin D3 offers a comprehensive treatment for problems issuing from osteoporosis, as it is intended to improve the bioavailability of both active ingredients to produce a pharmacological effect in less time.

The vitamin D3 shield does increase its stability, since the blend of the emulsifier, antimicrobial and antioxidant agents contributes various functions which aid it in maintaining stability. According to the results that were obtained, the vitamin D3 rating is constant throughout the period in which it normally degrades.

The components of the pharmaceutical formulation also increase the bioavailability of risedronate sodium and the Phosal 50 PG contributes a phosphatidylcholine which is a component of cell membranes, and which therefore serves as a carrier to aid in the absorption of risedronate sodium.

1. A stable pharmaceutical composition consisting of therapeutically effective doses of risedronate and cholecalciferol that is useful in the inhibition of bone resorption, characterized by a granular core containing risedronate or one of its pharmaceutically acceptable salts and at least one excipient; cholecalciferol, Vitamin D3 with shielding sprayed onto the risedronate granules, a first coating of the risedronate granules sprayed with shielded cholecalciferol; pharmaceutically acceptable excipients for the preparation of a tablet from sprayed risedronate granules with shielded cholecalciferol and coatings, and an enteric coating on the tablet.

2. The composition according to claim 1, which is characterized because it comprises between 10 to 40% of the weight of the risedronic acid composition or its pharmaceutically acceptable derivatives, its salts, anfiolates and hydrates; between 0.001 to 0.05% of the weight of the composition of cholecalciferol, Vitamin D3; from 50 to 80% of the weight of the composition of at least one emulsifier, from 2 to 6% of the weight of the composition of at least one disintegrating agent, from 2 to 10% of the weight of the composition of at least one diluting agent, 0.1 to 2% by weight of the composition of at least one binding agent, from 0.2 to 0.5% of the weight of the composition of a first enteric coating, from 3 to 5% of the weight of the composition of a second enteric coating, from 0.25 to 5% of the weight of the composition of at least one lubricating agent and a sufficient amount of one or more solvents or solvent mixture as the dissolution medium.

3. The composition according to claim 1, wherein the diluent is selected from the group including cellulose PH 102, microcrystalline cellulose PH 101, Ludipress, Prosolv 90; the disintegrating agent is selected from the group consisting of croscarmellose sodium, crospovidone, and sodium starch glycolate; the binder is selected from the group consisting of polyvinylpyrrolidone K 30, povidone PK 90, copovidone, hydroxypropyl methylcellulose, ethyl cellulose; the emulsifying agent is selected from the group consisting of Phosal 50 PG, phosphatidylcholine in soy lecithin, propylene glycol, sunflower mono- and diglycerides and ascorbyl palmitate; the composition comprising an enteric coating is selected from NS Enteric and sodium alginate, Acryl-eze™, methacrylic acid copolymer, Sureteric®, polymethacrylates, triethylcitrate, triacetin, and polyethylene glycol; and
solvents are selected from purified water, ethanol, propanol and mixtures thereof.

4. The composition according to claim 1, which is characterized because the risedronate granule composition includes risedronate sodium, a disintegrating agent selected from the group consisting of croscarmellose sodium, microcrystalline cellulose and povidone K30.

5. The composition according to claim 1, which is characterized because the cholecalciferol shield consists of an emulsifier selected from the group comprised of Phosal, phosphatidylcholine in soy lecithin, propylene glycol, sunflower mono- and diglycerides and ascorbyl palmitate and ethanol as solvent.

6. The composition according to claim 1, which is characterized because the cholecalciferol solution is sprayed on the granulated risedronate sodium, and subsequently coated with a suspension of NS Enteric coating.

7. The composition according to claim 1, which is characterized because the tablet also includes one or more excipients selected from croscarmellose sodium, microcrystalline cellulose, povidone K30, Phosal and NS Enteric agent and a lubricant, such as magnesium stearate.

8. The composition according to claim 1, which is characterized because the coating consists of a methacrylic acid copolymer.

9. A process for the preparation of the composition according to claim 1, characterized because it includes the following stages:
a) PREPARE THE GRANULE COMPOSITION
b) PREPARE THE VITAMIN D3 SHIELD
c) COAT THE GRANULE WITH AN ENTERIC LAYER
d) FORM THE CORE OF THE TABLET; AND
e) COAT THE TABLET-CORE AND SHIELDED VITAMIN D3 WITH AN ENTERIC LAYER.

10. The process according to claim 1, which is characterized because the preparation of THE GRANULE COMPOSITION consists of the following steps:
a) Weigh, mix and granulate risedronate sodium, croscarmellose sodium, microcrystalline cellulose PH 102 and polyvinylpyrrolidone K 30 with purified water.
b) Sift using No. 14 mesh and dry in oven until less than 3.0% moisture is reached.

11. The process according to claim 1, which is characterized because the preparation of the VITAMIN D3 SHIELD consists of the following steps:
a) Dissolve vitamin D3 in Phosal 50 PG and subsequently dilute with ethyl alcohol, mix until it forms a clear, yellow solution.
b) Introduce the dry risedronate granules into a fluid bed basin, and permeate with the vitamin D3 spray solution at a temperature between 25° to 30° C.

12. The process according to claim 1, which is characterized because the preparation of the GRANULE ENTERIC COATING consists of the following steps:
a) Prepare a suspension of NS Enteric in purified water at 10% solids, stir until completely incorporated and sift using No. 50 mesh.
b) Atomize the above suspension in the granules at a temperature of 25° to 35° C., dry the granules in a fluid bed and sift using No. 18 mesh.

13. The process according to claim 1, which is characterized because the GRANULE CORE FORMATION consists of the following steps:
a) Sift using No. 20 mesh: microcrystalline cellulose PH 102, croscarmellose sodium and mix with the granules from step No. 6 for 5 minutes.
b) Lubricate with magnesium stearate, previously sieved through No. 30 mesh, for 3 minutes.
c) Compress in 4 mm hexagonal press at 200 mg ±5% of weight.

14. The process according to claim 1, which is characterized because the preparation of the ENTERIC COATING FOR THE TABLET-CORE AND SHIELDED VITAMIN D3 consists of the following steps:
a) Prepare a coating suspension with Acryl-eze™ and triethyl citrate in purified water at 20% solids.
b) Coat the tablets with the Acryl-eze™ suspension at a temperature of 30° to 35° C. in a perforated drum.
c) Prepare the coating suspension with Opadry® FX in purified water to 6.5% solids.
d) Coat the tablets with the Opadry® FX suspension at a temperature of 38° to 43° C. in a perforated drum.

15. The composition according to claim 1, which is characterized because the cholecalciferol has a stability greater than 93% after 4 weeks at 30° C./65% RH, 40° C./75% RH, and Cycled 40° C./75% RH and RH-Cooling (2° C.-8° C.).

16. The composition according to claim 1, which is characterized because the dissolution of risedronate was no more than 10.0 at acid pH and is 100% at physiological pH at 10 minutes.
